(19) United States
(12) Patent Application Publication
(10) Pub. No.: US 2022/0079159 A1
(43) Pub. Date: Mar. 17, 2022

(54) COVERS WITH BIOACTIVE SURFACE COATINGS FOR USE ON DOOR KNOBS, LATCHES AND HANDLES

(71) Applicants: Dennis Hackemeyer, Forest, VA (US); Mark Sisson, Forest, VA (US)

(72) Inventors: Dennis Hackemeyer, Forest, VA (US); Mark Sisson, Forest, VA (US)

(21) Appl. No.: 17/212,609

(22) Filed: Mar. 25, 2021

Related U.S. Application Data
(60) Provisional application No. 63/084,097, filed on Sep. 28, 2020.
(60) Provisional application No. 63/000,359, filed on Mar. 26, 2020.

(51) Int. Cl. A01N 59/16; A61L 2/26; A61L 2/232; A61L 2/238; A01N 25/10; B32B 7/12; B32B 27/40; B32B 25/20; B32B 25/08; B32B 1/00; E05B 1/00

(52) U.S. Cl. CPC A01N 59/16; A61L 2/26; A61L 2/232; A61L 2/238; A01N 25/10; A61L 2101/02; B32B 27/40; B32B 25/20; B32B 25/08; B32B 1/00; E05B 1/0061; B32B 7/12

(57) ABSTRACT

Certain embodiments described herein are directed to door knob covers, latch covers, urinal handle covers and other covers that can reversibly couple to an underlying structure. In some examples, the cover comprises a bioactive material that can kill or inactivate bioorganisms. The bioactive material can be a photocatalyst and may also comprise one or more transition metals. Methods of preventing or reducing the spread of infections using the covers are also described.

### PRIORITY APPLICATIONS

This application claims priority to and the benefit of each of U.S. Provisional Application No. 63/000,359 filed on Mar. 26, 2020 and U.S. Provisional Application No. 63/084,097 filed on Sep. 28, 2020, the entire disclosure of which is hereby incorporated herein by reference.

### TECHNOLOGICAL FIELD

Certain configurations described herein are directed to door knob covers comprising one or more bioactive materials present on a surface and/or embedded in the door knob covers.

### BACKGROUND

Door knobs are often contacted by many different people. Different users can transfer germs or other materials to the door knob. These germs or materials can then be transferred to a subsequent user.

### SUMMARY

Certain aspects described herein are related to door knob covers that can be coupled to a door knob. The door knob cover can be placed over the door knob and held in place through a friction fit such that turning of the door knob cover acts to turn the door knob. The door knob cover may comprise one or more of a surface coating, a coating with an embedded material or both. The exact configuration of the door knob cover can depend, at least in part, on the shape and size of the door knob with illustrative configurations permitting the door knob cover to be stretch over and/or round the door knob for placement. For example, the cover can be used in connection with round door knobs or projections on slide latches. Various examples are described to illustrate some of the many different configurations of the door knob cover.

In certain embodiments, a door knob cover comprising a substrate, an optional adhesive layer, a carrier support material and surface coating is described. In some embodiments, a first adhesive layer (when present) is coupled to the substrate, and a carrier support material is coupled to the first adhesive layer or the substrate when the first adhesive layer is absent. The substrate can be configured to stretch around and contact an outer surface of a door knob. In certain examples, the surface coating is coupled to the carrier support material and comprises a bioactive material to inactivate or kill bioorganisms that contact the surface coating. In some configurations, the door knob cover is configured to stretch and fit around a circumference of a door knob and rotate the door knob when the door knob cover is rotated by an end user.

In certain examples, the bioactive material comprises at least one of titanium, silver, copper and zinc. In other examples, the carrier support material comprises a polyurethane or a silicone. In other examples, the bioactive material comprises a photocatalyst comprising titanium, and wherein the bioactive material comprises at least one additional transition metal.

In some embodiments, the door knob cover further comprises a second bioactive material embedded in the carrier support material, e.g., embedded in a polyurethane or silicone material. In some instances, the bioactive material and the second bioactive material comprise different transition metals. In other configurations, the first adhesive layer comprises a residue free adhesive.

In some examples, the substrate is configured as a sleeve with the first adhesive layer and the carrier support material present on an outer surface of the sleeve. In some configurations, the door knob cover is optically transparent. In other embodiments, the substrate comprises a silicone.

In another aspect, a door knob cover comprises a substrate, an optional a first adhesive layer coupled to the substrate, and a carrier support material coupled to the substrate or the optional first adhesive layer when present. In certain embodiments, the carrier support material comprises embedded bioactive material to inactivate or kill bioorganisms that contact the door knob cover. In some configurations, the door knob cover is configured to stretch and fit around a circumference of a door knob and rotate the door knob when the door knob cover is rotated by an end user.

In certain embodiments, the bioactive material comprises at least one of titanium, silver, copper and zinc. In other embodiments, the carrier support material comprises a polyurethane. In further embodiments, the bioactive material comprises a photocatalyst comprising titanium, and wherein the bioactive material comprises at least one additional transition metal. In other embodiments, the door knob cover comprises a second bioactive material embedded in the carrier support material. In some instances, the bioactive material and the second bioactive material comprise different transition metals. In certain embodiments, the first adhesive layer comprises a residue free adhesive. In other embodiments, the substrate is configured as a sleeve with the adhesive layer and the carrier support material present on an outer surface of the sleeve. In certain examples, the door knob cover is optically transparent. In other embodiments, the substrate comprises a silicone.

In another aspect, a door knob cover comprises a substrate comprising an open end and a closed end, an optional first adhesive layer coupled to the substrate, a carrier support material coupled to the first adhesive layer (when present) or the substrate, and a surface coating coupled to the carrier support material. In certain embodiments, the surface coating comprises a bioactive material to inactivate or kill bioorganisms that contact the surface coating, wherein the door knob cover is sized and arranged to cover outer surfaces of the door knob when the open end of the door knob cover is placed over the door knob. In some embodiments, the substrate can be configured to stretch around and contact an outer surface of a door knob

In other embodiments, the door knob cover comprises a removable flap in the substrate. In some embodiments, the substrate is configured as a sleeve with the closed end and the open end. In some examples, the door knob cover is optically transparent. In other examples, the substrate comprises a silicone.

In an additional aspect, a method comprises placing one or more of the door knob covers described herein onto a door knob to facilitate transfer of infectious organisms, infectious virus, infectious viral agents or infectious viral particles from a human to the placed door knob cover, wherein the placed door knob cover comprises a bioactive material to kill or inactivate the transferred infectious organisms, infectious virus, infectious viral agents or infectious viral particles. In some embodiments, the virus that is transferred and inactivated is a coronavirus.

In another aspect, a method comprises reducing community spread of an infection by placing one or more of door knob covers described herein onto a door knob to facilitate transfer of infectious organisms, infectious virus, infectious viral agents or infectious viral particles from a user to the placed door knob cover. In certain examples, the placed door knob cover comprises a bioactive material to kill or inactivate the transferred infectious organisms, infectious virus, infectious viral agents or infectious viral particles so successive humans touching the placed door knob cover do not become infected by the infectious organisms, infectious virus, infectious viral agents or infectious viral particles. In some embodiments, the virus that is transferred and inactivated is a coronavirus.

In another aspect, a method of treating a human infected with an infectious organism, an infectious virus, an infectious viral agent or an infectious viral particle comprises administering to the infected human a therapeutic to treat the infection, and reducing spread of the infection from the infected human to third parties by placing one or more of the door knob covers described herein onto a door knob to facilitate transfer of the infectious organisms, infectious virus, infectious viral agents or infectious viral particles from the infected human to the placed door knob cover. In certain embodiments, the placed door knob cover comprises a bioactive material to kill or inactivate the transferred infectious organisms, infectious virus, infectious viral agents or infectious viral particles so successive humans touching the door knob cover do not become infected by the infectious organisms, infectious virus, infectious viral agents or infectious viral particles. In other embodiments, the therapeutic is an antimicrobial agent. In some examples, the therapeutic is an antiviral agent.

In another aspect, a kit comprises a therapeutic to treat human infected with an infectious organism, an infectious virus, an infectious viral agent or an infectious viral particle, and a door knob cover comprising a bioactive material to kill or inactivate any infectious organisms, infectious virus, infectious viral agents or infectious viral particles transferred from the infected human so successive humans touching the door knob cover do not become infected by the infectious organisms, infectious virus, infectious viral agents or infectious viral particles. In certain embodiments, the therapeutic is an antimicrobial agent. In other embodiments, the therapeutic is an antiviral agent. In some examples, the kit comprises written or electronic instructions for using the therapeutic to treat the infection and using the door knob cover to prevent or reduce spread of the infection.

In another aspect, a slide latch cover comprises a substrate, an optional first adhesive layer coupled to the substrate, a carrier support material coupled to the first adhesive layer (when present) or the substrate, and a surface coating coupled to the carrier support material. In some examples, the surface coating comprises a bioactive material to inactivate or kill bioorganisms that contact the surface coating, wherein the slide latch cover is configured to stretch and fit around a boss of a slide latch that is contacted by a user to slide the slide latch.

In an additional aspect, a slide latch cover comprises a substrate, an optional first adhesive layer coupled to the substrate, and a carrier support material coupled to the first adhesive layer (when present) or the substrate. In certain embodiments, the carrier support material comprises embedded bioactive material to inactivate or kill bioorganisms that contact the slide latch cover, wherein the slide latch cover is configured to stretch and fit around a boss of a slide latch that is contacted by a user to slide the slide latch.

In another aspect, a urinal handle comprises a substrate, an optional first adhesive layer coupled to the substrate, a carrier support material coupled to the first adhesive layer (when present) or the substrate, and a surface coating coupled to the carrier support material. In certain embodiments, the surface coating comprises a bioactive material to inactivate or kill bioorganisms that contact the surface coating, wherein the urinal handle is configured to stretch and fit around a handle of urinal that is contacted by a user to flush the urinal.

In an additional aspect, a urinal handle comprises a substrate, an optional first adhesive layer coupled to the substrate and a carrier support material coupled to the first adhesive layer (when present) or the substrate. In certain embodiments, the carrier support material comprises embedded bioactive material to inactivate or kill bioorganisms that contact the urinal handle, wherein the urinal handle is configured to stretch and fit around a handle of a urinal that is contacted by a user to flush the urinal.

Additional aspects, embodiments, examples and configurations are described in more detail below.

### BRIEF DESCRIPTION OF THE SEVERAL VIEWS OF THE DRAWINGS

FIGS. 1A, 1B and 1C are photographs of a door knob cover coupled to a door knob and FIG. 1D is an illustration showing a door knob cover, in accordance with some examples;

FIG. 2A is an illustration of a door knob cover, and FIG. 2B is an exploded view of a section of the door knob cover showing a bioactive material present in a surface coating, in accordance with some examples;

FIG. 3A is an illustration showing a door knob cover, and FIG. 3B is an exploded view of a section of the door knob cover showing a bioactive material embedded in a carrier support material of a surface coating, in accordance with some examples;

FIG. 4A is an illustration of a door knob cover, and FIG. 4B is an exploded view of a section of the door knob cover showing a bioactive material present in a surface coating and embedded in a carrier support material, in accordance with some examples;

FIGS. 5A, 5B, 5C, 5D and 5E show shapes for different carrier support materials;

FIG. 6 is an illustration showing a door knob cover comprising a bioactive material, in accordance with some examples;

FIG. 7 is an illustration showing a door knob cover with a memory that is retained in a curve shape, in accordance with some embodiments;

FIG. 8 is an illustration of a sleeve that can be cut into multiple door knob covers;

FIG. 9 is an illustration of a kit including a door knob cover and a door knob, in accordance with certain examples;

FIG. 10 is an illustration of a slide latch comprising a projection or boss that can be covered with a cover as described herein;

FIGS. 11A and 11B are illustrations showing a cover placed on a handle of a urinal, in accordance with some embodiments;

FIG. 11C is an illustration of a sleeve that can be placed on a handle;

FIG. 12 is a table showing reduction of a coronavirus using an article with a bioactive material; and

FIG. 13 is a table showing reduction of E. coli using an article with a bioactive material.

### DETAILED DESCRIPTION

Certain articles are described below in connection with door knob covers that can be placed around a door knob. The door knob may be present on many different devices including, but not limited to, residential exterior and interior doors, commercial exterior and interior doors, closet doors, and other devices that include, for example, a round door knob. As noted herein, the door knob cover is typically configured as a cylindrical device that can be stretched around a door knob and couples to the door knob through a friction fit. In some instances, the door knob cover may frictionally engage the door knob and not couple to the door knob through the use of an adhesive. The door knob cover is typically non-electronic and does not comprise any moving parts but does include one or more bioactive materials as noted in more detail below.

The exact materials used in the door knob covers and other devices described herein may vary in thickness, hardness, composition etc. For example, the door knob covers or other devices described herein may have a Shore A hardness of 40-80, more particularly a Shoe A hardness of 50-70. The exact wall thickness of the devices may also vary from about 0.04 inches to about 0.08 inches, more particularly about 0.05 inches to about 0.07 inches. In some embodiments where silicone or other polymeric materials are present in the door knob covers or other devices, the Shore A hardness of the device may vary from about 50-70 when a wall thickness of the device is about 0.05 inches to about 0.07 inches.

In some examples, the door knob covers described herein can comprise a bioactive material that can kill or inactivate bioorganisms. The term “bioorganism” is intended to include, but is not limited to, bacteria, fungi and bacterial and fungal spores as well as any viruses or portions thereof, e.g., any membrane components or other components of the bacteria, fungi or virus that may be secreted. Illustrative bioorganisms that are targeted include gram positive and gram negative bacteria, Staphylococcus, Escherichia coli, Propionibacteria, Corynebacteria, dermobacteria, and micrococci, Tinea, Candida, flu virus, adenoviruses and other bacterial, fungi and viruses. The bioactive material may also be effective to inactivate or render non-toxic secreted proteins and materials such as endotoxins or other toxins.

In certain embodiments, the door knob covers described herein may comprise one or more surface coatings or layers. In some examples, the surface coating may comprise a bioactive material on an outer surface of the surface coating. In other instances, the surface coating may comprise an embedded bioactive material. In additional examples, the surface coating may comprise a bioactive material on an outer surface of the surface coating and may also comprise an embedded bioactive material. For example, as the bioactive material on the outer surface breaks down or is otherwise removed by contact, the embedded bioactive material may still be present to kill or inactivate bioorganisms. In some examples, the surface coating or layer may comprise a carrier material or support that can receive the bioactive material on its surface or can permit embedding of the bioactive material. While not wishing to be bound by any one configuration, the surface coating is generally a non-transfer surface coating such that no or little material is transferred to a user contacting the surface coating with their hands, fingers or other body part. For example, bioorganisms can be transferred from the user to the surface coating where they are inactivated, killed or oxidized by the bioactive material.

In certain embodiments, the bioactive material in the surface coating may be photoactivatable and/or photorechargeable to permit continuous use and reuse of the door knob cover. For example, the surface coating can be exposed to infrared, visible, ultraviolet or light of other wavelengths to activate the bioactive material in the surface coating such that the bioactive material can function as a photocatalyst. For example, the bioactive material may comprise photocatalytic titanium dioxide or other photocatalytic transition metal materials. Once activated, the bioactive material can, for example, oxidize groups or constituents on bioorganisms to inactivate and/or kill them. The bioactive material may be photorechargeable by exposing the bioactive material to additional light for an activation period, e.g., 10 seconds or more, 20 seconds or more, 30 second or more, 1 minute or more, etc. Reactivation recharges the bioactive material for addition use. While the bioactive material may be exposed to light for a suitable period, actual recharging of the material can occur quickly, e.g., within a few microseconds, milliseconds, etc.

In some configurations, the bioactive material may comprise a metal or a material which can release ions, e.g., within the carrier support. For example, the bioactive material can be a transition metal or a transition metal containing material that includes one or more transition metals which can be present in ionic form and/or complexed with one or more ligands. Without wishing to be bound by any one configuration, the transition metal may be present in different forms in the carrier material including free ions and complexed ions. In some examples, the transition metal may be any one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition materials being desirable to use and with ionized forms of the transition metals being desirable for use in some instances. In other instances, the bioactive material may comprise two or more different transition metals each of which can independently be present as free ions or complexed with a ligand or other groups. In other instances, the bioactive material may comprise three or more different transition metals each of which can independently be present as free ions or complexed with a ligand or other groups. Where the bioactive material is a photocatalyst, the bioactive material may comprise one or more transition metals. Where the bioactive material is embedded in a surface coating or a carrier support material, the bioactive material desirably can release transition metal ions which can bind to and/or be taken up by the bioorganisms.

In certain embodiments, the transition metal material can be selected to oxidize constituents or groups present on the bioorganisms to kill or inactivate the bioorganisms. For example, the transition metal or transition metal material may function as a photocatalyst and can be activated by exposure of the surface coating to ultraviolet or visible light or light of other wavelengths. Subsequent to initial use, the transition metal or transition metal material can be photo-charged (or photo-recharged) by exposing the surface coating to additional ultraviolet or visible light or light of other wavelengths. The exact interval where at least 50% of the material remains in an activated form may vary from a few days to a few weeks or even a few months. At any time, a certain amount of the bioactive material may be present in an active state to photocatalyze the received bioorganisms while some portion of the bioactive material may be present in an inactive state. Recharging may be performed, for example, when the amount of bioactive material in the active state drops below a certain percentage, e.g., 50%, 40%, 30%, 20% or even 10%.

In certain configurations, the bioactive material can also be present on top of the carrier support material as a separate surface coating. For example, the support material may comprise embedded bioactive material and additional bioactive material may be present as a separate surface layer or surface coating on top of the carrier support material comprising the embedded bioactive material. In some cases, bioactive material can migrate from the carrier support material into the outer surface coating or layer to replenish the bioactive material as it is consumed or leaches off. In other instances, the embedded bioactive material does not migrate but can remain active within the carrier support material to kill and/or inactivate bioorganisms. Where a surface coating of bioactive material is deposited on top of a layer comprising the embedded bioactive material in the carrier support material, the surface coating may comprise a transition metal or a transition metal containing material that includes one or more transition metals which can be present in ionic form and/or complexed with one or more ligands. The transition metal bioactive material deposited on top of the carrier support material may be present in different forms in the carrier material including free ions and complexed ions. In some examples, the transition metal present on top of a carrier support material may comprise one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, and mercury, with non-radioactive transition material being desirable to use and with ionized forms of the transition metals being desirable for use in some instances. In some instances, the transition metal bioactive material deposited on top of the carrier support material may comprise two or more different transition metals each of which can independently be present as free ions or complexed with a ligand or other groups. In other instances, the transition metal material deposited on top of the carrier support material may comprise three or more different transition metals each of which can independently be present as free ions or complexed with a ligand or other groups.

In certain examples, the carrier support material of the door knob covers described herein typically is selected to be able to withstand physical contact of the door knob covers to surfaces. For example, the carrier material may be a polymeric material that can be disposed on a substrate in a desired shape and using suitable methods, e.g., printing, spraying, coating, dip coating, rolling or using other methods. As noted herein, a bioactive material of the surface coating can be present on or in the carrier material (or both) and used to inactivate or kill bioorganisms. In some embodiments, the carrier support material can be selected such that it retain the bioorganisms within the surface coating, e.g., prevents transfer of the bioorganisms back to a second user contacting the articles. In some embodiments, the carrier material may comprise one or more thermoplastics or one or more thermosetting materials. For example, polyurethanes, polyacrylates, and copolymers comprising polyurethanes, polyacrylates or other polymeric materials that are optically transparent when placed on a substrate can be used. In other instances, the carrier support material may be a polyester, an epoxy resin, a polyimide, a silicone resin, a vinyl ester resin, a polycarbonate, a polyetherimide, a polypropylene, a polyphenylene oxide, a polyphenylene sulfide or other resins or materials that are desirably optically transparent, though the carrier support materials may be opaque or partially opaque if desired.

In certain embodiments, the surface coating may be hard, soft, non-compressible, compressible or elastomeric depending on the end use and configuration of the final article. In some examples, the carrier support material may be elastic and optionally comprise one or more elastomeric materials. For example, upon depression of the door knob cover by a user, the door knob cover spring back to an initial position after removal of the depressing force. In some instances, rubber, natural rubber, synthetic rubber or other rubber based materials may be present in the carrier support material.

In other instances, the bioactive material may form clusters on top of the carrier support material with open space present between the clusters. In such instances, it may be desirable to include embedded bioactive material in the carrier support material as well in case bioorganisms do not contact any of the surface clusters of the bioactive material.

In certain embodiments, the door knob covers described herein are typically placed on top of a door knob and can be designed to permit viewing of the underlying door knob. For example, the entire door knob cover may be produced using materials which are generally transparent, e.g., over visible wavelength ranges, such that viewing of the underlying door knob is permitted. The door knob cover need not transmit 100% of the light but can be generally transparent enough so underlying material of the door knob is viewable using the naked eye. Even though the door knob cover can be optically transparent, they may be colored if desired. Alternatively, the door knob cover may be colorless. If desired, the door knob cover may also be optically opaque and/or include printed text, designs or other indicia on one or more surfaces.

In certain embodiments, the layers of the door knob cover may comprise fibers, elastomers or other materials to alter the overall properties of articles. For example, elastomeric fibers may be present to permit stretching of the door knob cover around a door knob. Further, additional materials may be present to provide touch indicia such as Braille, raised letters or numbers or other features.

In certain configurations, the door knob covers described herein generally comprise a substrate upon which the surface coating (or other layers) is placed. In certain embodiments, suitable substrates that can be used with the surface coating described herein may be optically transparent, printed or may be opaque if desired. In certain examples, the exact material used in the substrate can vary depending on the intended use environment of the article. In some examples, the substrate may comprise a paper, a fabric, a metal, a non-metal, a plastic, a ceramic, a glass, a fiberglass, a stone, a wood, a rubber, a foam, a textile, cardboard, a vinyl material, concrete, asphalt, leather, suede, a polymeric material, silicone or other materials. In embodiments where papers are used, the paper may be acid-free or may be designed to be present in its use environment for a desired period without substantial degradation. In examples where a fabric is used, the fabric may be a woven fabric, a non-woven fabric, a polyester fabric such as, for example, a draw textured yarn (DTY) polyester fabric, a polyester-copolymer fabric and other fabrics commonly used to receive inks and colorants using printing techniques, lithographic techniques or other techniques. For example, polyester DTYs are effective to absorb and retain inks and other colorants. In addition, polyester DTYs can permit even distribution of the inks or colorants to provide desirable indicia. Illustrative DTY's can be found, for example in U.S. Patent Publication No. 20110008563 filed on Jul. 9, 2009, the entire disclosure of which is incorporated herein by reference. Where the substrate is a ceramic, the ceramic may be, for example, aluminum oxide, yttrium oxide, cerium oxide, beryllia, zirconia, a carbide, a boride, a nitride, a silicide or other ceramic materials. Where the substrate is a glass, the glass may be colored, non-colored, opaque, transparent or may include variable areas having different properties. If desired, the glass may include reinforcing fibers or other materials to strengthen the physical properties of the glass. Where the substrate is a stone, wood, rubber, foam or other material, the material may be porous such that physical indicia can be imparted to the material. If the material is highly porous, then it may be desirable to reduce the porosity of the material by first disposing an agent on the material that can occupy some of the pores of the material. In some embodiments, the substrate may be a plastic material such as, for example, a thermoplastic material or a thermosetting material. In other embodiments, silicone or polyurethane materials may be used or present in the substrate.

In some examples, the substrate may comprise a polyolefin material that is optically transparent. For example, the substrate material may comprise a polyethylene, a polyethylene copolymer, a polyvinyl chloride, a polyvinyl chloride copolymer, a polypropylene or other polyolefins that are optically transparent at least to some degree. In some embodiments, the polyolefin may be non-porous or substantially non-porous and be configured to retain the surface coating and optionally other materials on a surface of the substrate. For example, while the surface coating may include some porosity to permit bioorganisms to penetrate into the surface coating, the substrate generally can be non-porous or fully consolidated such that surface coating materials do not penetrate into the substrate. Illustrative substrates are commercially available from many different sources including, but not limited to, those from the PhotoTex® Group Inc. (Boardman, Ohio), Fusion Digital (Washington, Utah), Yupo (Chesapeake, Va.), Granwell (West Caldwell, N.J.), Superior Fabrics (Pompano Beach, Fla.), Worthen Industries (Nashua, N.H.) and other commercial sources. In some instances, the substrate can be flexible, rigid, semi-rigid, compressible or may have other physical properties as desired.

In some examples, the overall shape and thickness of the various layers may vary as desired and depending on the intended use of the article. In some examples, the carrier support material layer may comprise a thickness, for example, of about 0.5 mm to about 5 mm. Where a surface coating of bioactive material is present on top of the carrier support material layer, the surface coating thickness may be, for example, about 0.1 mm to about 1 mm. The overall thickness of the substrate can vary from about 0.1 mm to about 5 mm. The width and length of the door knob covers can depend on the end use and size of the door knob and illustrative values are discussed below. The thickness of the bioactive material can be as little as 1-2 crystals, e.g., 7-10 nm or less.

In certain embodiments, one or more protective layers, materials or coatings may be present on the door knob covers described herein. The protective layer, material or coating may be present between two or more other components of the articles as desired or within any one or more layers. In some examples, the protective layer, material or coating may be present on top of the active surface layer or coating. For example, in applications where the articles are used outside, a UV protective material, color fast material or other materials may be present on top of the active surface coating or mixed with it to protect it. The protective coating, material or layer can be porous to permit bioorganisms to be transferred from a user's hand or other body part to the underlying active surface coating or layer for inactivation, oxidation and/or killing. In other examples, the protective layer or material may be present on top of the substrate and used to render the substrate color fast or protect any ink or other colorant on the substrate from photobleaching, UV degradation or degradation due to other means. Suitable materials for use as a protective layer or coating include, but are not limited to, acrylates such as, for example, trimethylpropaneacrylate, epoxyacrylate, urethaneacrylate and other acrylates. Other polymeric materials including polyolefins, nanoparticles and the like may also be present as protective coatings.

In certain embodiments, the door knob covers described herein can optionally include an adhesive layer between the substrate and a release liner. The adhesive layer can be designed to adhere the door knob cover to an underlying door knob. In some examples, the adhesive can be a residue free adhesive such that removal of the door knob cover from the underlying door knob does not leave behind any adhesive on the surface of the underlying door knob. Illustrative adhesives include but are not limited to thermoplastic adhesives and thermosetting adhesives. For example, the adhesive may comprise one or more of adhesives or residue-free adhesives that are commercially available from 3M, Henkel, Shell Chemical Company, Kuraray Company and other commercial suppliers of adhesives. In some examples, the adhesive may comprise rubber or other elastomer and be a residue-free adhesive. In other examples, the adhesive can be a silicon based adhesive such as, for example, an organopolysiloxane adhesive. In some examples, the adhesive can include one or more cross-linkable groups such as, for example, an isocyanate group, an unsaturated hydrocarbon group, a sulfo group, a sulfhydryl group, an alkoxy group, a hydroxyl group, and other groups that can be cross-linked. In some embodiments, the adhesive can be used in combination with a crosslinking agent to facilitate cross-linking and/or curing of the adhesive. In some embodiments, the adhesive can include one or more materials, polymers or copolymers including, but not limited to, styrene block polymers such as, for example, a styrene and styrene/diene copolymer (SBS, SIS, SBR), a styrene/ethylene/butylene copolymer (SEBS) or a styrene/ethylene/propylene/styrene copolymers (SEPS), acrylate copolymers, a polyester urethane copolymer, an ethylene acrylate copolymer, a butyl rubber copolymer; a natural rubber copolymer; an ethylene/propylene copolymer; an ethylene/vinyl acetate copolymers, EPDM/PP, NR/PP, EVA/PVDC and NBR/PP, polyurethanes, polyether esters and polyether amides based copolymers or materials. Additional materials and groups can also be used to prepare the adhesive including, but not limited to, homo- or copolymers of 1,3-butadiene, 2-methyl-1,3-butadiene (isoprene), 1,3-pentadiene, 2-ethyl-1,3-butadiene, 2-propyl-1,3-butadiene, 2-isopropyl-1,3-butadiene, 2-hexyl-1,3-butadiene, 2,3-dimethyl-1,3-butadiene, 2,3-diethyl-1,3 -butadiene, 2-methyl-1,3-pentadiene, 2-methyl-1,3-hexadiene, 2-methyl-1,3-octadiene, 2-methyl-1,3-decadiene, 2,3-dimethyl-1,3-pentadiene, 2,3-dimethyl-1,3-hexadiene, 2,3-dimethyl-1,3-octadiene and 2,3-dimethyl-1,3-decadiene, 2-methyl-1,3-cyclopentadiene, 2-methyl-1,3-cyclohectadiene, 2,3-dimethyl-1,3-cyclopentadiene, 2,3-dimethyl-1,3-cyclohexadiene, 2-chloro-1,3-butadiene, 2,3-dichloro-1,3-butadiene, 1-fluoro-1,3-butadiene, 2-chloro-1,3-pentadiene, 2-chloro-1,3-cyclopentadiene and 2-chloro-1,3-cyclohexadiene. In some embodiments, isoprene, polyisoprene or isoprene derivatives or polyisoprene derivatives may also be used in the adhesive. If desired, the adhesive may be a pressure sensitive adhesive. In certain examples, the adhesive can be crosslinkable to the substrate using light, heat, a catalyst, an activator or other suitable materials and/or processes.

In certain configurations, an adhesive layer can be present between any two or more component or layers of the door knob covers. For example, an adhesive layer can be present between the carrier support material and the underlying substrate, between the carrier support material and any bioactive surface coating or between other layers that may be present in the articles.

In some examples, a release liner may be present on a surface of the door knob cover that is to be coupled to an underlying door knob. The release liner is typically an inert material, e.g., a paper, plastic, rubber, etc. that is used to cover the adhesive layer prior to use of the door knob cover. The release liner may comprise, for example, Kraft paper, clay coated paper, machine glazed paper, a polyethyleneterephthalate film, a polyethylene film, a polypropylene film, and other films produced using polyolefin materials.

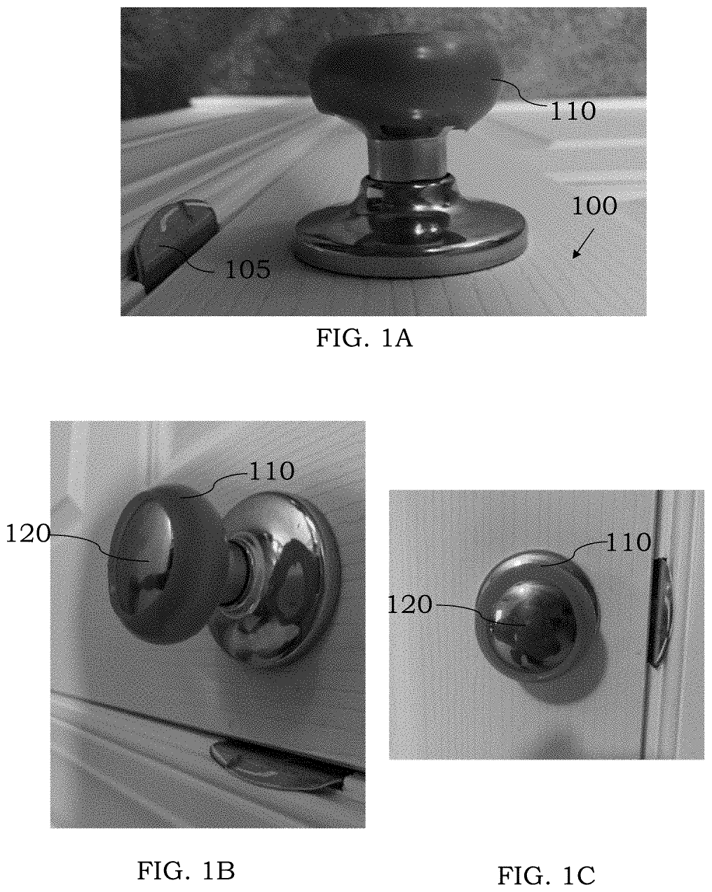

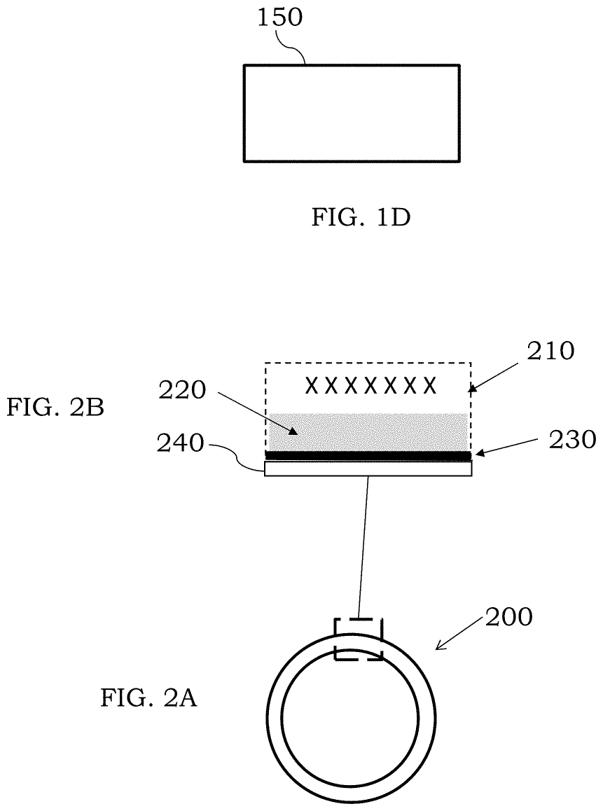

In certain configurations, photographs of a placed door knob cover 110 on a door knob 120 are shown in FIGS. 1A, 1B and 1C. A door 100 and a door striker plate 105 are shown for reference. The door knob cover 110 is shown as being stretched around a circumference of the door knob 120 with a central portion of a front surface of the door knob 120 being exposed. As noted in more detail below, most or all contact surfaces of the door knob 120 could instead be covered be a door knob cover if desired. FIG. 1D shows one configuration of a door knob cover that is configured as a sleeve 150 with open ends and having a sleeve length of about ⅞ inches or ¾ inches. If desired, the sleeve 150 can be longer (or shorter) and an end user can cut the door knob covers to length as desired. An outer surface of the sleeve 150 may comprise a bioactive material as noted in more detail below. The sleeve 150 can be slid over an open end of a door knob, e.g., prior to attachment of the door knob or after attachment of the door knob, depending on the door knob configuration, and held to the door knob using friction or an optional adhesive.

Referring to FIGS. 2A and 2B, one illustration of certain components and materials that are present in a selected section of a door knob cover are shown. The door knob cover 200 comprises a surface coating 210, a carrier support material 220, an adhesive layer 230, and a substrate 240. An optional additional adhesive layer and an optional release liner can also be present if desired. In this illustration, the bioactive material is present in the surface coating 210 (see FIG. 2B), which can be sprayed, dip coated, curtain coated, roller coated, printed, brushed or otherwise deposited on the carrier support material 220. If desired, two or more layers of the surface coating 210 can be sprayed onto the support material 220 as individual layers. The carrier support material 220 can be any of those materials mentioned above or other suitable materials. The adhesive layer 230 acts to retain the carrier support material 220 to the underlying substrate 240. When present, the additional adhesive layer coupled to the substrate 240 can retain the door knob cover 200 to a surface of the door knob when in use. The adhesive layer 230 and any additional adhesive layer can comprise the same or different materials as desired and may be any of the illustrative adhesive materials mentioned herein or other suitable adhesive materials. The substrate 240 is typically optically transparent (but can be optically opaque) and may be any of those illustrative materials discussed herein. In some instances, the substrate 240 may comprise an elastomeric or stretchable material that can be stretch around an outer surface of a door knob. While certain layers are shown in FIG. 2B as comprising the same thickness, this arrangement is not required or even typical. The adhesive layer 230 tends to be much thinner than the substrate 240 or the carrier support material 220. The bioactive material in the surface coating 210 typically comprises one or more transition metals or transition metal materials including, but not limited to, those comprising titanium, zinc, copper, silver or other transition metals mentioned herein. The bioactive material in the surface coating 210 may be present in ionic form, chelated or bound to other groups or both. In some instances, the bioactive material of the surface coating 210 can be photo-activated by exposure to ultraviolet light or visible light (or both) and can be photo-recharged upon re-exposure to ultraviolet light or visible light (or both). In other examples, the bioactive material in the surface coating 210 can function as a photocatalyst to kill or inactivate bioorganisms that contact the surface coating 210.

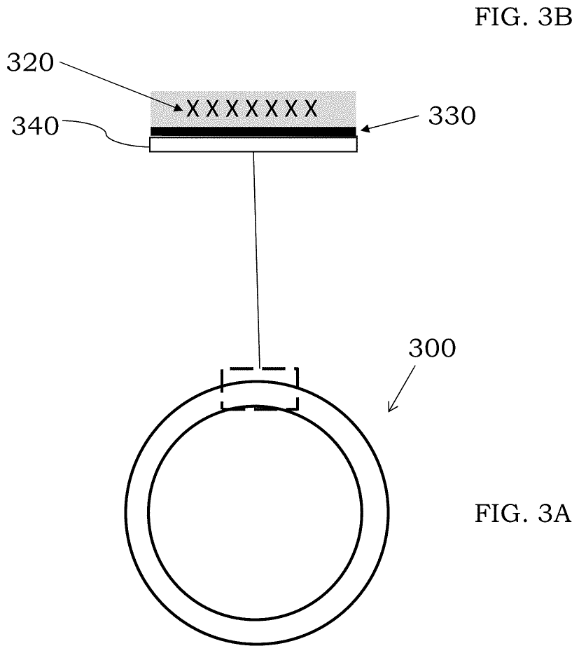

In certain configurations, other material arrangements for the door knob covers are also possible. Referring to FIGS. 3A and 3B, another illustration of certain components and materials that are present in a door knob cover is shown. The door knob cover 300 comprises a surface coating 320 comprising a carrier support material and a bioactive material, an adhesive layer 330, a substrate 340, and an optional additional adhesive layer and an optional release liner. In this illustration, the bioactive material is embedded within the carrier support material that is present in a surface coating 320, e.g., the carrier support material acts as the surface coating 320, which can be sprayed, dip coated, curtain coated, roller coated, printed, brushed or otherwise deposited on the underlying adhesive layer 330. The carrier support material in the coating 320 can be any of those materials mentioned above or other suitable materials. The adhesive layer 330 acts to retain the carrier support material 320 to the underlying substrate 340. When present, the additional adhesive layer can retain the door knob cover 300 to a surface of the door knob when in use. The adhesive layer 330 and any additional adhesive layer can comprise the same or different materials as desired and may be any of the illustrative adhesive materials mentioned herein or other suitable adhesive materials. The substrate 340 is typically optically transparent, but can be optically opaque, and may be any of those illustrative materials discussed herein. While certain layers are shown in FIG. 3B as comprising the same thickness, this arrangement is not required or even typical. The adhesive layer 330 tends to be much thinner than the substrate 340 or the surface coating 320. The bioactive material embedded in the carrier support material present in the surface coating 320 typically comprises one or more transition metals or transition metal materials including, but not limited to, those comprising titanium, zinc, copper, silver or other transition metals mentioned herein. The bioactive material in the surface coating 320 may be present in ionic form, chelated or bound to other groups or both. In some instances, the bioactive material of the surface coating 320 can be photo-activated by exposure to ultraviolet light or visible light (or both) and can be photo-recharged upon re-exposure to ultraviolet light or visible light (or both). In some examples, the bioactive material in the surface coating 320 can function as a photocatalyst to kill or inactivate bioorganisms that contact the surface coating 320.

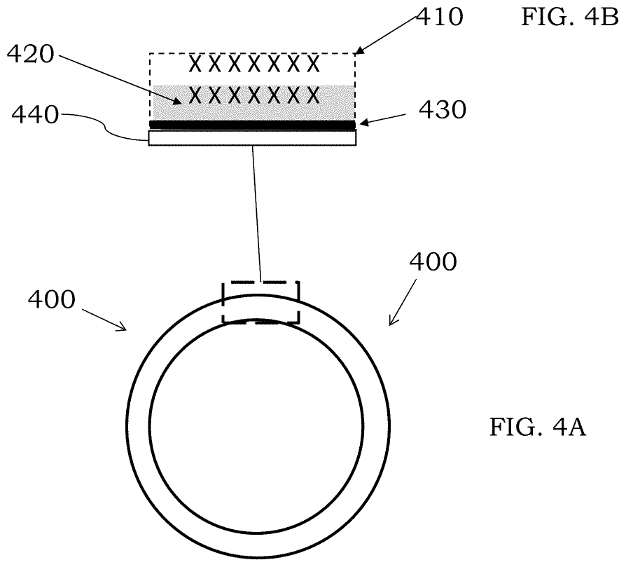

Referring to FIGS. 4A and 4B, an illustration is shown where a bioactive material is present in both a surface coating and is embedded in a carrier support material that underlies the surface coating. The door knob cover 400 comprises a surface coating 410 with a bioactive material, a carrier support material 420 (with a bioactive material), an adhesive layer 430, a substrate 440, and may comprise an optional additional adhesive layer and an optional release liner. In this illustration, the bioactive material is present in the surface coating 410, which can be sprayed, dip coated, curtain coated, roller coated, printed, brushed or otherwise deposited on the carrier support material 420. In addition, the carrier support material 420 also comprises an embedded bioactive material which may be the same or may be different than the bioactive material of the surface coating 410. The carrier support material 420 can be any of those materials mentioned above or other suitable materials. The adhesive layer 430 acts to retain the carrier support material 420 to the underlying substrate 440. When present, the additional adhesive layer can retain the door knob cover to the door knob when in use. The adhesive layer 430 and any additional adhesive layer can comprise the same or different materials as desired and may be any of the illustrative adhesive materials mentioned herein or other suitable adhesive materials. The substrate 440 is typically optically transparent, but can be optically opaque, and may be any of those illustrative materials discussed herein. While certain layers are shown in FIG. 4B as comprising the same thickness, this arrangement is not required or even typical. The adhesive layer 430 tends to be much thinner than the substrate 440 or the carrier support material 420. The bioactive material in the surface coating 410 and in the carrier support material 420 typically comprises one or more transition metals or transition metal materials including, but not limited to, those comprising titanium, zinc, copper, silver or other transition metals mentioned herein. The bioactive material in the surface coating 410 and in the carrier support material 420 may be present in ionic form, chelated or bound to other groups or both. In some instances, the bioactive material of the surface coating 410 and the carrier support material 420 can be photo-activated by exposure to ultraviolet light or visible light (or both) and can be photo-recharged upon re-exposure to ultraviolet light or visible light (or both). In some instances, the bioactive material in the surface coating 410 and in the carrier support material 420 can independently function as a photocatalyst to kill or inactivate bioorganisms that contact the door knob cover 400.

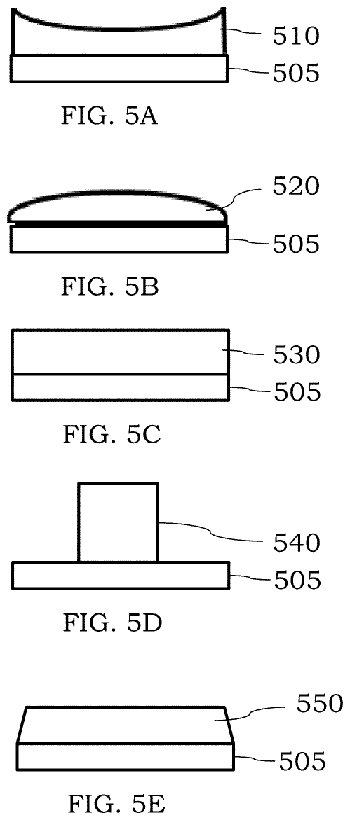

While the carrier support material is shown in FIGS. 2B, 3B and 4B as being generally planar or flat, this shape is not required. For example, a concave shape 510 (FIG. 5A), an half-ellipse shape 520 (FIG. 5B), a rectangular shape 530 (FIG. 5C), a square shape 540 (FIG. 5D), a trapezoidal shape 550 (FIG. 5E) or other geometric shapes for the carrier support layer present on a substrate 505 could instead be present and used to produce the door knob covers described herein. Similarly, the substrate may comprise many different shapes to form a door knob cover. Circular, elliptical, square, rectangular or substrates with other geometric shapes can be present. The substrate can first be formed prior to application of the various layers, or the various layers can be applied to a generally planar substrate which is then formed into a door knob cover. Where the door knob cover takes the form of a sleeve, one end of the sleeve can be closed and a second end of the sleeve can be open. The closed end of the sleeve can prevent touching of outer surfaces of the door knob, and the open end of the sleeve permits stretching of the door knob cover around the door knob during installation. In some instances, a flap may be present which can be flipped down to permit usage of a key or turning of a lock and then flipped back into place to cover the keyhole or lock when not being used. The flap may permit access to the underlying door knob features while at the same time preventing contact of the underlying door knob features when the door knob is being turned during normal use.

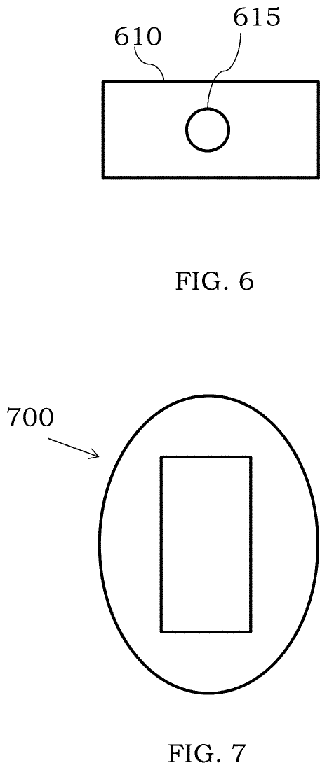

In some embodiments, some portion of the door knob cover may be open or exposed to permit viewing of the underlying door knob. FIG. 6 shows a door knob cover 610 that comprises an aperture or opening 615 which permits viewing or access of a certain portion or area of the door knob (not shown) underlying the door knob cover. For example, the door knob may comprise a keyhole, lock button, etc. which can be exposed through the opening 615 to permit a user to access this feature of the door knob. Further, the cover 610 may comprise a plurality of separate apertures or openings to permit viewing of different sections of the underlying door knob.

In certain examples, the door knob cover may have a “memory” which acts to retain the door knob cover in a desired shape or arrangement. One illustration is shown in FIG. 7, where the door knob cover 700 comprises a generally circular arrangement to permit placement of the door knob cover 700 over an end of the door knob. The door knob cover 700 is slid over the door knob end and positioned at a desired site on the door knob. One or more of the layers of the door knob cover 700 may have the memory to retain the door knob cover in the shape even where the door knob cover 700 is not coupled to a door knob. For example, the substrate of the door knob cover may be formed into a desired shape, e.g., using molding, thermoforming or other means, to provide a memory to the door knob cover 700. Alternatively, the door knob cover 700 can be present as a planar sheet which can be wrapped around at least some portion of a door knob. In the illustration shown in FIG. 7, the inner shape of the door knob cover 700 is generally rectangular while the outer shape of the door knob cover is generally elliptical to provide a more user friendly gripping surface. These shapes may generally remain even where the door knob cover 700 is not coupled to a door knob. If desired, the door knob cover may be heated to shrink the materials and enhance the fit between the door knob cover and the door knob. In other configurations, an inner shape of the door knob cover may be round and include an outer diameter that is 5% less, 10% less, 15% less, 20% less or eve 25% less than an outer diameter of the door knob so a tight fight between the door knob cover and door knob exists.

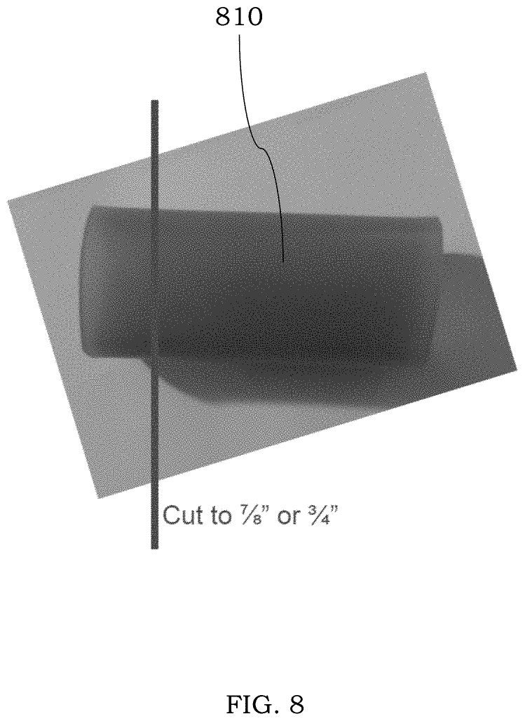

In some embodiments, a plurality of door knob covers coupled to each other can be preset and used to provide individual door knob covers. Referring to FIG. 8, a sleeve 810 is shown that can be cut into individual segments or door knob covers. The sleeve 810 can be sized and arranged such that 2, 3, 4, 5, 6, 7, 8, 9, 10 or more individual door knob covers can be cut from the sleeve

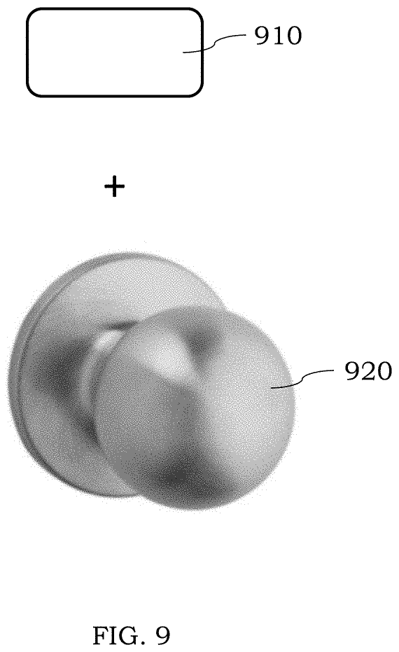

In certain embodiments, the door knob covers can be packaged in a kit with a door knob. One illustration is shown in FIG. 9, where a door knob cover configured as a sleeve 910 that may be present in a kit with a door knob 920. Written or electronic instructions may be present in the kit to facilitate installation and use of the door knob 920 and the door knob cover 910. If desired, door knob covers of varying size may be present in a kit to permit a user to select an appropriate size cover to place onto a door knob.

In certain embodiments, the door knob sleeve may comprise, for example, a length of about 0.5 inches to about 1.25 inches and a width of about 0.5 inches to about 4 inches. Where the door knob cover takes the form of a sleeve, a wall thickness of the door knob cover may be, for example, about 0.1 inches to about 0.5 inches. The wall thickness need not be the same at all areas around the sleeve if desired.

The articles and various layers described herein can also be used with additional materials including primers, e.g., titanium dioxide primer layers, colorants, inks, luminescent coatings, surfactants and other materials as desired. Crosslinkers such as amides or other materials can also be used to facilitate rapid curing of the layers or the layers can be cured without the use of any crosslinkers. In some embodiments, one or more of a halogenated phenol, a phenoxy phenol, a hydroxyphenyl ether, a halogenated phenoxy, e.g., fluorinated, chlorinated or brominated phenoxy compounds, polyhexamethylene biguanide (PHMD), PHMD chloride, PHMD fluoride, PHMD bromide, PHMD hydrochloride, Microban® materials, halogenated phenols such as, for example, 5-chloro-2-(2,4-dichlorophenoxy) phenol, chloro-2-(2,4-dichloro)phenol, and chloro-2-(2,4-dichlorophenoxy)phenol, Triclosan, Irgansan DP300, CH3635, Ster-zac, Lexol 300, trichloro-2-hydroxydiphenyl ether, plant oils such as, for example, tea tree oil, mint oil, leleshwa oil, sandalwood oil, clove oil, lavender oil, nigella sativa (Black cumin) oil, onion, garlic and combinations thereof can also be present in the surface coating or carrier support material or both. In some instances, the surface coating, carrier support material or both may include one or more materials commercially available from Environ (Rochester Hills, Mich.), Microban (Huntersville, N.C.), or Oxititan (Pompano Beach, Fla.) or other producers of antimicrobial ingredients.

Certain embodiments described herein can be used in methods and device to reduce infections and community spread of infections. The methods can desirably use one or more of the door knob covers described herein.

In some embodiments, a method of reducing infections comprises placing one or more of the door knob covers described herein onto another article or device to facilitate transfer of infectious organisms, infectious virus, infectious viral agents or infectious viral particles from a user to the door knob cover or surface. A bioactive material on the placed door knob cover can inactivate and/or kill the infectious virus, infectious viral agents or infectious viral particles to prevent infection of a subsequent user who contacts the door knob cover or surface.

In another embodiment, a method of reducing community spread of an infection comprises placing one or more of the door knob covers described herein onto another article or device facilitate transfer of infectious organisms, infectious virus, infectious viral agents or infectious viral particles from a user to the door knob cover or surface. A bioactive material on the placed door knob cover can inactivate and/or kill the infectious virus, infectious viral agents or infectious viral particles to prevent community spread of the infectious virus, infectious viral agents or infectious viral particles.

In another example, a method of treating a person infected with an infection while reducing spread of the infection from the infected person comprises placing a door knob cover comprising one or more bioactive materials on a corresponding receptive article and administering to the infected person in need of treatment one or more antiviral drugs, antimicrobial drugs or anti-parasitic drugs or combinations thereof. The drug administration can treat the infected person while the placed door knob cover can reduce spread of the infection from the human being treated to third parties. For example, the method can reduce spread by killing or inactivating of infectious organisms, infectious virus, infectious viral agents or infectious viral particles that have been transferred to the article using one or more bioactive materials on the door knob cover.

In certain examples, the methods and door knob covers described herein can be used to prevent or reduce the spread of a virus including double-stranded DNA viruses, a single-stranded DNA virus, a double-stranded RNA virus, a single stranded RNA virus, a single-stranded RNA retrovirus, a double-stranded DNA retrovirus and other viruses including either double-stranded DNA or RNA or single stranded DNA or RNA or hybrid DNA-RNA nucleic acid. Specific types of viruses include, but are not limited to, a picornavirus, a coronavirus, a rhinovirus, an adenovirus, an enterovirus, an influenza virus, a human parainfluenza virus, a human respiratory syncytial virus, a metapneumovirus, a retrovirus, a norovirus, a rotavirus, a herpes virus, a poxvirus, a reovirus, an orthomyxovirus, a rhabdovirus, a parvovirus and other viruses that can infect mammals such as humans or other animals. As noted in more detail below, the devices are particularly effective at reducing active levels of coronaviruses such as, for example, coronavirus 229E, coronavirus NL63, coronavirus 0C43, coronavirus HKU1, MERS-CoV, SARS-CoV and SARS-CoV-2 (COVID-19). In some instances, at least 95% of the coronavirus transferred to the door knob cover surface can be killed or inactivated by the bioactive material within 30 minutes after transfer to the door knob cover surface. In other instances, at least 95% of the coronavirus transferred to the door knob cover surface can be killed or inactivated by the bioactive material within 60 minutes after transfer to the door knob cover surface. In some embodiments, at least 95% of the coronavirus transferred to the door knob cover surface can be killed or inactivated by the bioactive material within 120 minutes after transfer to the door knob cover surface. In some instances, at least 99% of the coronavirus transferred to the door knob cover surface can be killed or inactivated by the bioactive material within 30 minutes after transfer to the door knob cover surface. In other instances, at least 99% of the coronavirus transferred to the door knob cover surface can be killed or inactivated by the bioactive material within 60 minutes after transfer to the surface. In some embodiments, at least 99% of the coronavirus transferred to the door knob cover surface can be killed or inactivated by the bioactive material within 120 minutes after transfer to the door knob cover surface.

In certain embodiments, the methods and door knob covers described herein can be used to prevent or reduce the spread of infections from one or more bacteria, including but not limited to, Bacillus, Pseudomonas, Bacteroides, Bordetella, Brucella, Campylobacter, Chlamydia, Clostridium, e.g., Clostridium difficile, Corynebacterium, Enterobacter, Enterococcus, Escherichia, Haemophilus, Klebsiella, Lactobacillus, Legionella, Listeria, Micrococcus, Mycobacterium, Mycoplasma, Neisseria, Rickettsia, Salmonella, Shigella, Staphylococcus, Streptococcus, Vibrio, Yersinia and other bacteria commonly encountered in a clinical setting. In certain examples, spores such as those from Bacillus species or Clostridium species, e.g., Clostridium difficile, can be inactivated. In some embodiments, the bioactive material can be effective to kill or inactivate one or more of Actinobacteria, Bacteriodetes, Firmicutes, Propionibacteriaceae, Lactobacillaceae and Proteobacteria as these bacteria are commonly encountered in public setting such as public restrooms and surfaces therein. If desired, the bioactive material can also be selected to kill or inactivate fungal organisms such as those commonly encountered in athletic facility showers, e.g., Tinea, Trichophyton, Candida and other fungal organisms.

In certain embodiments, any of the door knob covers described herein can be printed by applying suitable materials to a surface using a printer. The printer may be, for example, an inkjet printer, digital printer, laser printer, etc.

In certain instances, the door knob cover described herein can be used in combination with an antimicrobial agent or therapeutic. Illustrative antimicrobial agents include, but are not limited to, a sulfonamide, a trimethoprim-sulfamethoxazole, a quinolone, a fluoroquinolone, a quinone, a penicillin, a cephalosporin, a Beta-lactam antibiotic, a Beta-lactamase inhibitor, an aminoglycoside, a tetracycline, a chloramphenicol, an erythromycin, a macrolide, a clindamycin, isoniazid, rifampin, a pyrazinamide, an ethionamide, amphotericin B, imidazole, triazole, ketoconazole, miconazole, itraconazole, fluconazole, ciclopirox olamine, haloprogin, tolnaftate, naftifine, terbinafine, chloroquinone, and hydroxychloroquinone. Other antibacterial and antifungal agents could also be used. Combinations of two or more of any of these antimicrobial agents can also be used in combination with the door knob cover described herein.

In some embodiments, the door knob cover described herein can be used in combination with one or more antiviral agents or therapeutics including, but not limited to, Abacavir, Acyclovir, Adefovir, Amantadine, Ampligen, Amprenavir, Arbidol, Atazanavir, Atripla, Balavir, Baloxavir marboxil, Biktarvy, Boceprevir, Cidofovir, Cobicistat, Combivir, Daclatasvir, Darunavir, Delavirdine, Descovy, Didanosine, Docosanol, Dolutegravir, Doravirine, Ecoliever, Edoxudine, Efavirenz, Elvitegravir, Emtricitabine, Enfuvirtide, Entecavir, Etravirine, Famciclovir, Fomivirsen, Fosamprenavir, Foscarnet, Fosfonet, a fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, a protease inhibitor, Pyramidine, Raltegravir, Remdesivir, a reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and combinations thereof.

In some embodiments, an antimicrobial agent can be used in combination with an antiviral agent and one or more of the door knob covers described herein. For example, the antiviral can be used to treat a viral infection, an antimicrobial can be used to treat a secondary bacterial infection and the door knob covers described herein can be used to prevent or reduce spread of the viral and/or antimicrobial infection to third parties.

In some embodiments, the door knob covers described herein can be dispensed in a vending machine or other devices to permit addition of the door knob covers to a door knob by an end user. For example, the door knob covers can be placed in public places such as offices, public, airports, etc. to permit a user to purchase and place the door knob covers on an door knob present in a public setting.

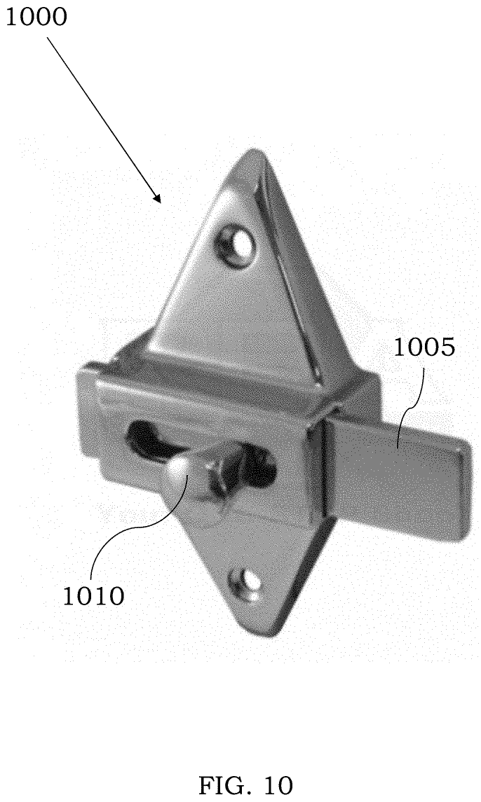

In certain examples, the door knob covers could instead be used to cover a sliding latch such as those commonly encountered in public bathroom stalls. For example, a door knob cover with a closed end can be slid over the latch button and retained through a friction fit. In such configurations, the length of the cover may be significantly lower than a length present when the door knob cover is used with a conventional door knob. Referring to FIG. 10, a sanitary slide bolt latch 1000 is shown that comprises a projection or boss 1010 that can be used to slide a latch 1005 inward and outward. A cover as described herein can be placed over the boss 1010 to prevent users from transferring germs or other materials to each subsequent users during use of the slide bolt latch 1000. The cover placed on the boss 1010 may include those materials described herein, e.g., may comprise a surface coating comprising a bioactive material to inactivate or kill bioorganisms that contact the surface coating and/or may comprise a carrier support material comprising embedded bioactive material to inactivate or kill bioorganisms. The latch may be present in many different settings including doors, bathroom stalls, locker rooms, showers or other settings where a latch is used to lock and unlock a door, window, container or other device.

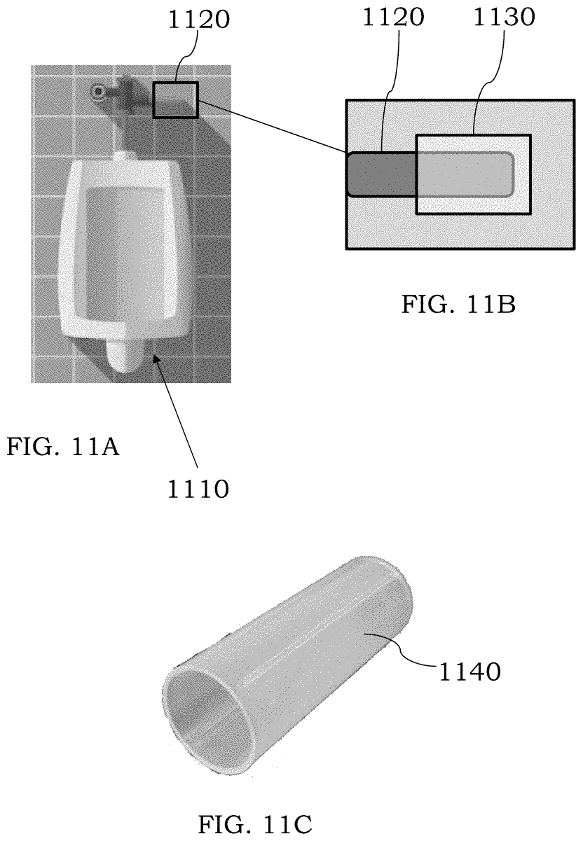

In certain embodiments, the covers can be placed on items other than door knobs and latches. For example, covers can be placed on toilet handles, urinal handles or other handles in a bathroom. Referring to FIG. 11A, a urinal 1110 is shown that includes a handle 1120. As shown in FIG. 11B, the handle 1120 has a cover 1130 placed on an end of the handle 1120. The cover 1120 can be configured similar to the covers described herein or instead could take the form of a sleeve with one end of the cover 1130 being closed. For example, a sleeve 1140 is shown in FIG. 11C that can be coupled to a urinal handle or other device. The cover 1130 can be produced from any of those materials described herein and typically is clear, e.g., may comprise a clear silicone or other clear polymer, to permit the viewer to see what is underneath the cover 1130. If desired, however, the cover 1130 could be opaque, colored or include texture. As noted herein, in certain configurations, a wall thickness of the cover 1130 or sleeve 1140 can vary from about 0.04 inches to about 0.08 inches, and a Shore A hardness of the cover 1130 or the sleeve 1140 can vary from about 40 to about 80. In some examples, the cover 1130 or the sleeve 1140 may include those materials described herein, e.g., may comprise a surface coating comprising a bioactive material to inactivate or kill bioorganisms that contact the surface coating and/or may comprise a carrier support material comprising embedded bioactive material to inactivate or kill bioorganisms.

Certain specific examples are described showing the articles and materials thereon can be used to kill or inactivate viruses and bioorganisms.

### EXAMPLE 1

A 8.5 inches by 11 inches sheet of material including a bioactive material comprising titanium dioxide doped with silver ions present in a surface coating was aseptically cut into 1″×1″ squares. Stainless steel control squares of the same size were ethanol sanitized and double rinsed in reverse osmosis prepared water and then autoclaved prior to use. Each of the test and control samples were placed into sterile Petri dishes using sterile forceps.

A stock vial of human coronavirus 229E (ATCC VR-740) was removed from cryo-storage and permitted to thaw. 0.010 mL aliquots were aseptically spread over the surface of each test and control square to ˜⅛ inch of the edge. Virus films were prepared in duplicate per test and control surface, per contact time (T=30 min, 1 hour, 2 hours and 4 hours). Control and test carrier were dried with Petri dish lids slightly ajar for 20 minutes at 24.7 degree Celsius, 36% relative humidity, Illuminance 1140 lux. Contact times were initiated when the control and test squares were visibly dry.

At the end of each contact time, the test and control carriers were aseptically transferred to tubes containing 2.0 mL of neutralizing solution (2% FBS EMEM). The carriers were vortexed for 30 seconds each to mechanically dislodged the microorganisms for enumeration. The inoculated sides of each carrier were further treater using a cell scraper to ensure adequate removal of the test viruses.

For cytotoxicity and neutralization effectiveness controls, one test and one control carrier each (with no virus film) were each aseptically transferred to neutralization tubes, and vortexed as described previously for the virus. The vortexed suspensions were serially diluted ten-fold in neutralizing solution, and selected dilutions were plated in quadruplicate onto the appropriate host cell monolayers (MRC-5, ATCC CCL-171) prepared to suitable confluency in multi-well trays. Virus control, cytotoxicity, neutralization validation, and sterility controls were performed concurrently. Virus reductions were calculated using the Spearman-Karber Method. Reference may be made to JIS Z 2801:2000. Antimicrobial Products—Test for Antimicrobial Activity and Efficacy. Japanese Standards Association. Tokyo, Japan.

No cytotoxicity was observed for the MRC-5 cells on the stainless steel control and tested squares.

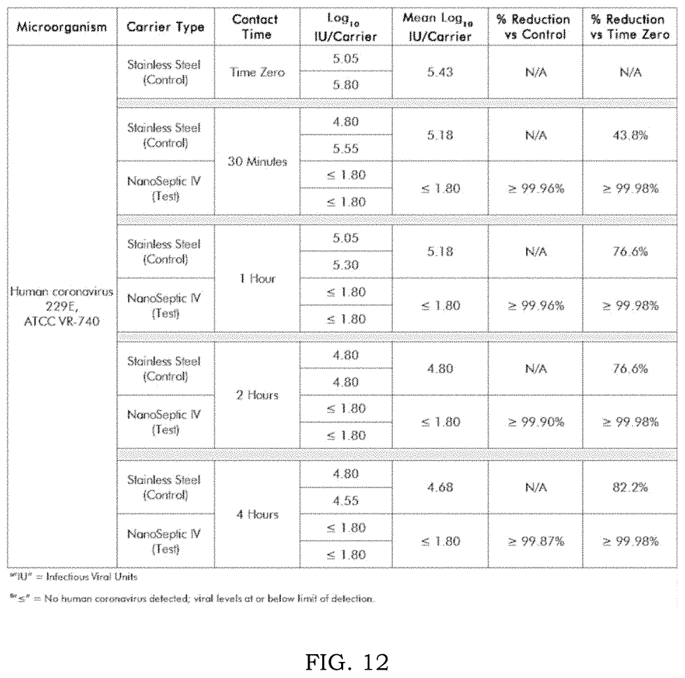

Referring to FIG. 12, as can be seen the test samples (labeled Nanoseptic IV), showed over a 99.96% reduction in viral for all measured times. In contrast, stainless steel control samples showed significantly less reduction at all measured times. These results are consistent with the tested samples being able to inactive the coronavirus and prevent infection of the MRC-5 human lung fibroblast cells.

### EXAMPLE 2

An article (2 inches by 2 inches) comprising a bioactive material comprising titanium dioxide doped with silver ions in a surface coating was tested for its ability to kill E. coli. using a modified ISO 22196 protocol. An overnight culture of E. coli cells (ATCC 8739) was diluted in sterile 1:500 Nutrient Broth. A sterile swab was dipped into the prepared test inoculum and used to inoculate each carrier via 13 passes (left to right=1 pass). Inoculated carriers were allowed to dry for 5 minutes followed by initiation of the contact time. Carriers were harvested after 5, 20, 60 and 120 minutes, vortexed to elute the viable bacteria and enumerated using standard dilution and pour plate techniques. Three replicates at each contact time were measured. Percent reduction was calculated as 100×(C−A)/C where A was the number of bacteria on the test carriers after the contact time and C is the number of bacteria on the control at time zero.

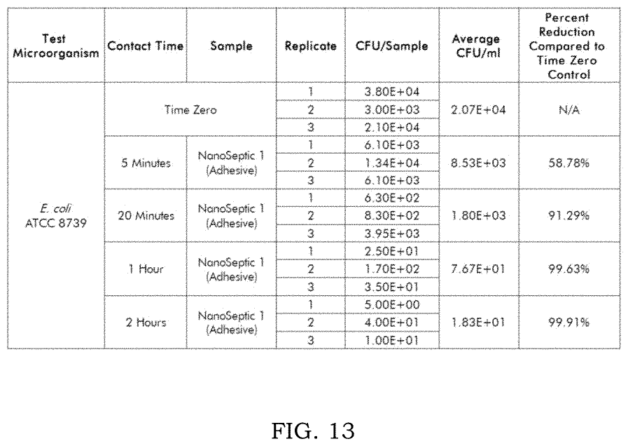

The results are shown in FIG. 13. At 20 minutes, a reduction in over 90% was observed. At 1 hour a reduction over 99% was observed. These results are consistent with the tested samples being able to kill the E. coli.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations.

1. A door knob cover comprising:
a substrate sized and arranged to stretch and fit around a door knob and held in place through a friction frit;
a first adhesive layer coupled to the substrate;
a carrier support material coupled to the first adhesive layer; and
a surface coating coupled to the carrier support material, the surface coating comprising a bioactive material to inactivate or kill bioorganisms that contact the surface coating, wherein the door knob cover is configured to stretch and fit around a circumference of a door knob and rotate the door knob when the door knob cover is rotated by an end user.

2. The door knob cover of claim 1, wherein the bioactive material comprises at least one of titanium, silver, copper and zinc.

3. The door knob cover of claim 2, wherein the carrier support material comprises a polyurethane.

4. The door knob cover of claim 1, wherein the bioactive material comprises a photocatalyst comprising titanium, and wherein the bioactive material comprises at least one additional transition metal.

5. The door knob cover of claim 3, further comprising a second bioactive material embedded in the polyurethane of the carrier support material.

6. The door knob cover of claim 5, wherein the bioactive material and the second bioactive material comprise different transition metals.

7. The door knob cover of claim 3, wherein the first adhesive layer comprises a residue free adhesive.

8. The door knob cover of claim 1, wherein the substrate is configured as a sleeve with the first adhesive layer and the carrier support material present on an outer surface of the sleeve.

9. The door knob cover of claim 8, wherein the door knob cover is optically transparent.

10. The door knob cover of claim 9, wherein the substrate comprises a silicone.

11. A door knob cover comprising:
a substrate;
a first adhesive layer coupled to the substrate; and
a carrier support material coupled to the first adhesive layer, the carrier support material comprising embedded bioactive material to inactivate or kill bioorganisms that contact the door knob cover, wherein the door knob cover is configured to stretch and fit around a circumference of a door knob and is held in place through a friction fit to rotate the door knob when the door knob cover is rotated by an end user.

12. The door knob cover of claim 11, wherein the bioactive material comprises at least one of titanium, silver, copper and zinc.

13. The door knob cover of claim 12, wherein the carrier support material comprises a polyurethane.

14. The door knob cover of claim 11, wherein the bioactive material comprises a photocatalyst comprising titanium, and wherein the bioactive material comprises at least one additional transition metal.

15. The door knob cover of claim 13, further comprising a second bioactive material embedded in the polyurethane of the carrier support material.

16. The door knob cover of claim 15, wherein the bioactive material and the second bioactive material comprise different transition metals.

17. The door knob cover of claim 13, wherein the first adhesive layer comprises a residue free adhesive.

18. The door knob cover of claim 11, wherein the substrate is configured as a sleeve with the adhesive layer and the carrier support material present on an outer surface of the sleeve.

19. The door knob cover of claim 18, wherein the door knob cover is optically transparent.

20. The door knob cover of claim 19, wherein the substrate comprises a silicone.

20-40. (canceled)
